(19) United States
(12) Patent Application Publication
(10) Pub. No.: US 2011/0098712 A1
(43) Pub. Date: Apr. 28, 2011

(54) Support arrangement for use in supporting a bone during a surgical operation

(75) Inventors: Eugene Sherry, Penrith (AU); Michael Egan, Blacktown (AU); Bob Lye, Brookvale (AU)

(73) Assignee: INTERNATIONAL PATENT OWNERS (CAYMAN) LIMITED, Grand Cayman (KY)

(21) Appl. No.: 10/553,652

(22) PCT Filed: Apr. 14, 2004
(86) PCT No.: PCT/AU04/00486

§ 371 Date: Aug. 23, 2006

(30) Foreign Application Priority Data
Apr. 14, 2003 (AU) 2003901738

(51) Int. Cl. A61F 5/00

(52) U.S. Cl. 606/87

(57) ABSTRACT

A support arrangement for use in supporting a bone during a surgical operation. The arrangement includes one or more supports located by a guide means, each support including means to fit around and support said bone during said operation.

### FIELD OF THE INVENTION

The present invention relates to a support for use in carrying out surgical procedures and relates in particular to a bone support to assist with removal of unwanted material from within a bone during forms of hip surgery.

The invention has been developed primarily for removing bonding and other material from within a femur during hip revision surgery, and it will be described hereinafter with reference to this application. However, it will be appreciated that the invention is not limited to this particular field of use and may be adapted to other surgical procedures.

### BACKGROUND OF THE INVENTION

Prior art hip replacement surgical techniques typically involve a surgeon firstly making a large initial incision so as to reveal the hip joint. The surgeon then typically manipulates the leg of the patient to dislocate the hip bone. This often requires the application of significant force to effect dislocation. Excessive manipulation and application of force may cause collateral damage to the patient, possibly resulting in post operative pain and/or an extended healing time. The head of the femur is then cut off at the neck. A cavity is reamed into the hip to accept a prosthetic acetabular cup (for example a LINK T.O.P. Acetabular Cup) and a prosthetic stem (for example a.LINK C.F.P. Hip Stem) is inserted into the femoral shaft. A bonding agent, such as surgical cement, is typically used to bond the prosthetic acetabular cup into the cavity in the hip and to bond the prosthetic stem to the femoral shaft. Typically, the surgeon aligns the cutting and reaming tools by eye, possibly resulting in minor misalignments. Hence, once the prosthetics are installed, there may be visually imperceptible misalignments between the prosthetic acetabular cup and the prosthetic femoral head. This may result in problems such as misalignment of the leg, incorrect leg length and/or incorrect soft tissue tension. Additionally, in the long term, misaligned prosthetic components may wear more quickly, giving rise to aseptic loosening of components and potentially necessitating early repetition of the surgery.

Due to prosthetic component wear over time also giving rise to aseptic loosening of components, many patients require repetition of the surgery. This repetition of surgery, or hip revision surgery, involves replacement of the prosthetic components. It is therefore necessary to remove the worn out components so that they can be replaced with new prosthetics. The removal techniques typically involve a surgeon removing or chipping the old cement away from within the femur and hip of the patient, so that the new prosthetics can be installed. While the surgeon is removing the old cement, there is a risk that the chipping will cause the bone to collapse outwardly and break. This can result in complications to the surgery. Any discussion of the prior art throughout the specification should in no way be considered as an admission that such prior art is widely known or forms part of the common general knowledge in the field.

### DISCLOSURE OF THE INVENTION

It is an object of the present invention to overcome or ameliorate at least one of the disadvantages of the prior art, or to provide a useful alternative.

It is an object of the present invention in its preferred form to provide a method and apparatus for more safely removing unwanted material from within the femur of a patient during hip revision or other surgery.

It is also desirable to provide a bone support which facilitates safe removal of cement from a femur which has been subject to a previously implanted prosthesis.

According to one aspect of the invention there is provided a bone support for use in supporting a bone during a surgical operation in which unwanted material is removed from within a cavity of the bone formation, the support including one or more support clamps located by a guide, each support including engagement means to fit around and support said bone during said operation.

Preferably, said support includes a surgical tool guide means to guide a cutting tool for removing said unwanted material.

Preferably, the or each support clamp including two arms hingedly connected at or adjacent their proximal ends, each of said arms including a respective one of a pair of opposed bone-supporting jaws, at its distal end.

Preferably, the support clamp includes an adjustment means for selectively moving said arms into and out of gripping engagement with the bone.

Preferably, the adjustment means is located at or adjacent said proximal ends of the arms. The adjustment means may include a shaft threadedly engaged through one said arm; a handle located at one end of said shaft; and an abutment portion located at the other end of said shaft such that relative rotation of said handle brings said abutment portion into and out of abutment with the other said arm thereby moving said gripping formations into and out of gripping engagement. The bone-supporting jaws are preferably concave to fit closely around the outer surface of the bone.

Preferably, a plurality of bone supports are positioned along the bone to fully support it along its length. Desirably, each bone support is aligned relative to the longitudinal axis of the bone cavity.

For preference, the surgical tool guide means includes a guide rail mounted to at least one of said arms wherein said guide rail is adapted to receive a guide display for displaying the orientation of said bone. Preferably, the guide display is a guide rod slidably mounted to the guide rail. In some embodiments the guide rail is movably mounted to at least one of the arms. In other embodiments the guide rail is fixedly mounted to at least one of the arms.

According to another aspect of the invention there is provided a bone support for use during removal of unwanted material from within a cavity of a bone formation during surgery, said support including a surgical support clamp adapted to engage an external surface of the bone formation, the clamp having a pair of elongated arms with opposed bone-supporting jaws at one end to engage the bone formation, a connecting arrangement by which the arms are connected together and are moveable relative to each other, and an arm moving arrangement associated with the arms by which the opposed jaws are adapted to be urged together to securely grip and support the bone formation.

It will be understood that the bone support of the preferred embodiments acts to limit the likelihood of a bone collapsing outwardly during the process of removing unwanted material from the bone cavity. The support may also assist in maintaining a structurally sound bone formation to receive an implant.

According to another aspect the present invention provides a surgical chisel for use in the method of the first aspect, said chisel including:
a shaft having a hollow portion adjacent a cutting end;
   an abutment portion at the other end of said shaft;

the hollow portion having tapering internal walls extending inwardly towards a central axis of the shaft to define a cutting edge at said cutting end.

Preferably, the hollow portion extends along a major portion of the length of said shaft. For preference, said shaft is generally circular in cross section.

In another aspect, the present invention provides a method of performing hip revision surgery on a patient, said method including the steps of:

a) exposing a femoral bone formation of said patient;

b) applying at least one support means as previously described to said femoral bone formation to support the structure of femoral formation; and

c) removing unwanted material from within said femoral bone formation. Preferably, the method further includes the step of:

d) extracting a first implant from within said femoral bone formation.

Even more preferably, the method further includes the step of:

inserting a second implant into said femoral bone formation.

Preferably also, the support means is a surgical clamp including two arms hingedly connected at substantially their proximal ends, each of the arms respectively including one of a pair of opposable gripping formations at its distal end.

More preferably step a) includes exposing the femoral bone of the patient through at 5 least two incisions adjacent the bone.

Even more preferably, step b) includes applying the gripping formations to the femoral bone formation through the at least two incisions adjacent the bone.

### BRIEF DESCRIPTION OF THE DRAWINGS

A preferred embodiment of the invention will now be described, by way of example only, with reference to the accompanying drawings in which:

FIG. 1 is a front view of a bone support clamp according to a preferred embodiment of the invention shown supporting a femoral bone formation;

FIG. 2 is a side view of the surgical clamp shown in FIG. 1;

FIG. 3 is an enlarged front view of an adjusting formation according to a preferred embodiment of the present invention;

FIG. 4 is a side view of the adjusting formation shown in FIG. 3;

FIG. 5 is an enlarged cut-away view of a guide rail according to a preferred embodiment of the present invention;

FIG. 6 is a front view of a surgical cutting tool according to a preferred embodiment of the present invention; and

FIG. 7 is a depiction of a femoral shaft illustrating a preferred embodiment of the invention shown supporting the structure of the femoral shaft.

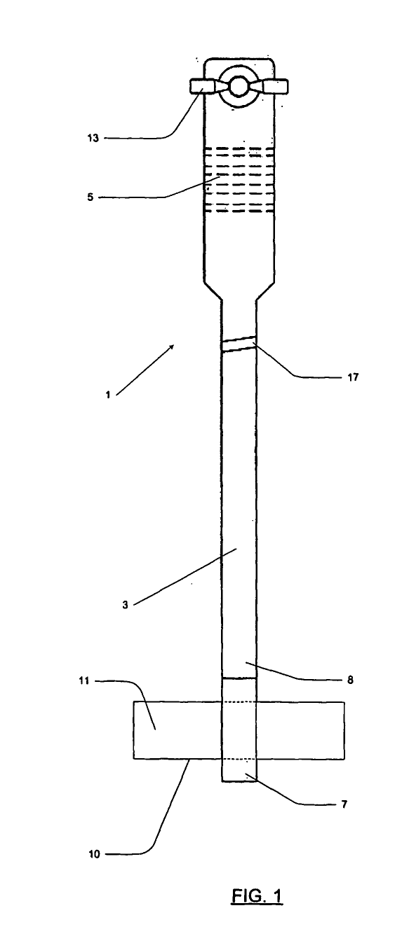

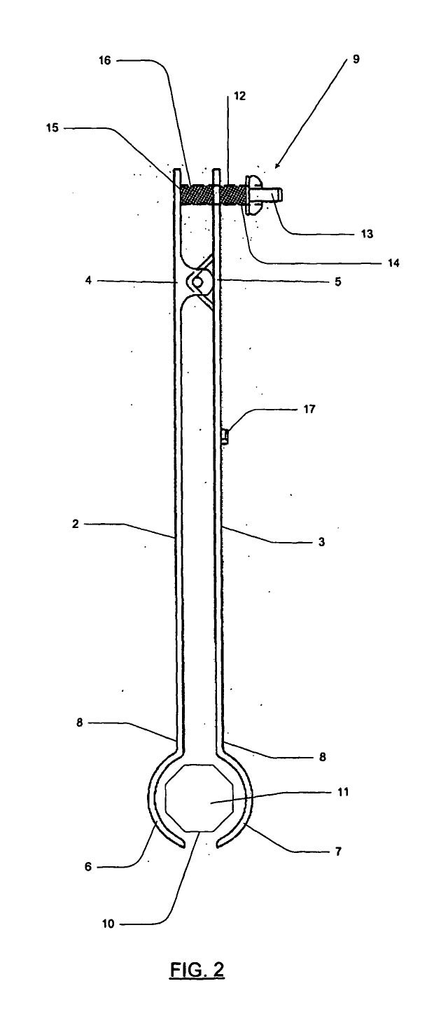

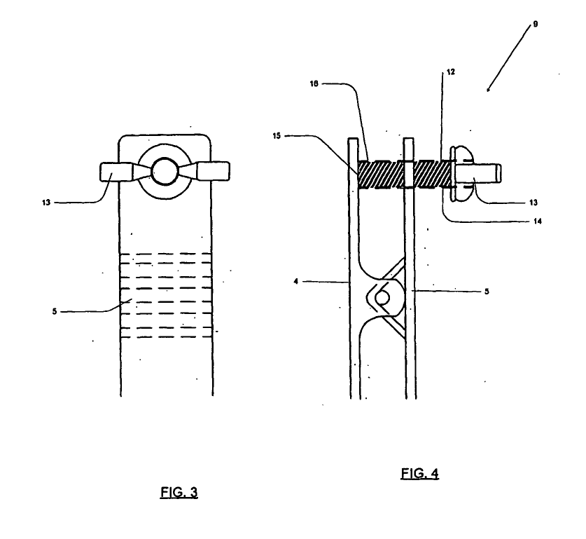

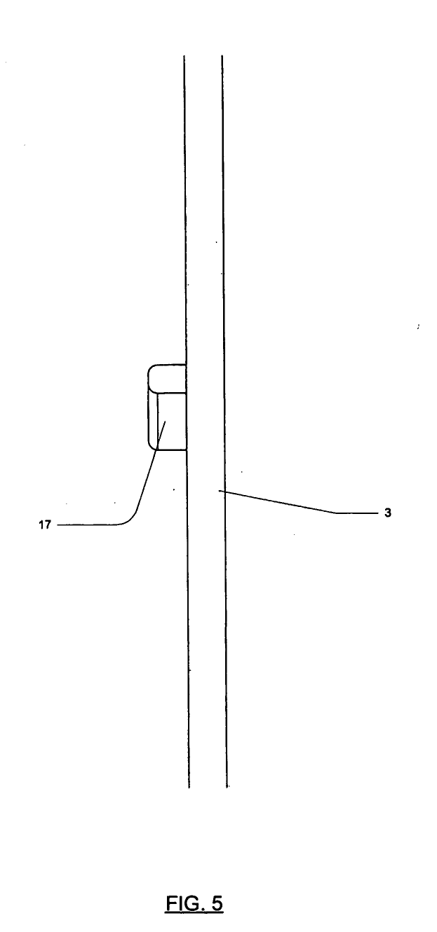

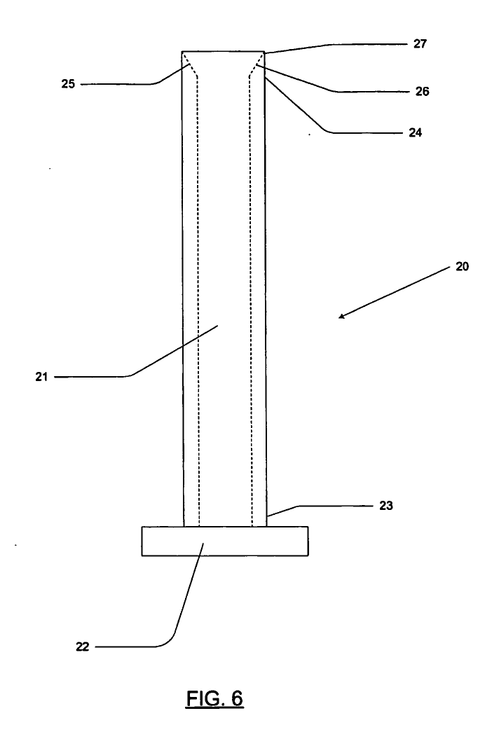

### PREFERRED EMBODIMENT OF THE INVENTION

Referring to the drawings, the support takes the form of a surgical clamp 1 which includes two elongated arms 2 and 3. The arms 2 and 3 are hingedly connected adjacent their proximal ends at 4 and 5. Each of the arms 2 and 3 includes a respective one of a pair of opposed bone-supporting, gripping formations, or jaws, 6 and 7 at the distal end 8. In this embodiment, the gripping formations, or jaws, 6 and 7, are concave. However, in other embodiments, the gripping formations may take other shapes. For example, in one preferred embodiment, they jaws are substantially flat. The jaws 6 and 7 have a lateral extent, relative to the arms 2 and 3. so as to engage a substantial portion of and along the length of the bone formation 11 to which the support is fitted to thereby support that portion, at least, against outward collapse.

The surgical clamp 1 includes an adjusting mechanism 9 for selectively moving the arms 2 and 3 into and out of gripping engagement with an outer surface 10 of the femoral bone formation 11. The adjusting mechanism 9 includes a threaded shaft 12 threadedly engaged through one of the arms 3. A handle 13 is located at one end 14 of the shaft 12 and an abutment portion 15 is located at the other end 16 of the shaft. Relative rotation of the handle 13 selectively moves the abutment portion 15 into engagement with the other arm 2, to thereby move the jaws 6 and 7 into gripping engagement with the femoral bone formation 11.

A surgical tool guide means, in the form of a guide rail 17, is either fixedly or adjustably mounted to arm 3 to guide a cutting tool for removing unwanted material such as glue or cement from within the femoral bone formation 11. The guide rail 17 is adapted to receive a guide display, in the form of a guide rod 19 (FIG. 7), which is slidably mounted to the guide rail 17 for displaying the orientation of the femoral bone formation 11 relative to one or more bone supports 1.

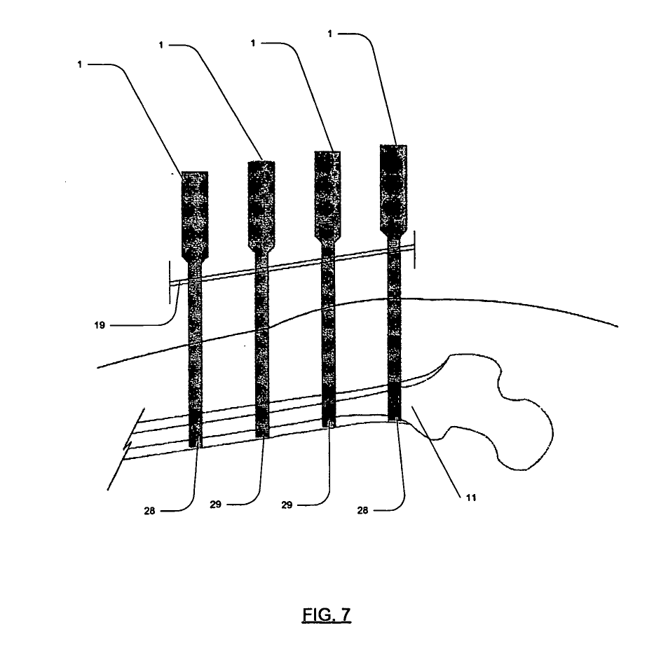

Referring to FIG. 6 there is shown a cutting tool in the form of surgical chisel 20. The chisel 20 includes a hollow shaft 21 and an abutment portion 22 at one end 23 of the shaft. A cutting end 24 is provided at the other end of the shaft 21. The cutting end has inwardly tapering walls 25 and 26 which define a cutting edge 27.

Referring to FIG. 7, in use, a number of the clamps 1 are inserted by the surgeon through incisions adjacent the femoral bone 11 of the patient and positioned with the bone-supporting jaws 6 and 7 around the surface bone formation 11 over a portion of its length. In this embodiment each pair of jaws 6 and 7 is applied to the bone formation 11 through separate respective incisions (not shown). The respective arms 2 and 3 of each of the supports which extend outside the patient are then adjusted by the respective handles 13 to bring the opposed jaws 6 and 7 into supporting engagement with the bone formation 11 along a portion of the length of the bone formation 11. Each support is aligned relative to each other and to the bone formation 11 by the guide rail 17. As each clamp is of the same length, the guide rail 17 extends generally parallel to and provides a guide as to the longitudinal axis of the bone formation. The rod 19 attached to the guide rail 17 can thus be used to indicate to the surgeon the position of the bone formation and to assist in guiding a surgical tool used for cleaning out the cavity of the bone formation. Once the bone is fully supported by the bone-supporting jaws 6 and 7, the surgeon can commence removal of cement or other unwanted material from the cavity of the bone formation 11 by using the chisel 20. It will be appreciated that some implants are not cemented or bonded into place and that the preferred embodiment of the invention may be used in either of those situations. The configuration of the chisel 20 allows waste material to be captured and withdrawn by means of the hollow shaft 21. Some examples of waste material include existing waste particles, elements of chronic inflammation and generated waste particles. The chisel is typically dimensioned so as to fit neatly within the bone cavity and allow alignment with the guide rod 19. It will be apparent to those in the art that a variety of other means can be used to remove unwanted material from the bone cavity provided the bone is adequately supported during removal.

While in some preferred embodiments the guide rail 17 is fixedly mounted to arm 3, in other embodiments it is movably mounted to the arm. For example, when used in the configuration as shown in FIG. 7, the outer surgical clamps 28 may have guide rails that are fixedly mounted, while the inner clamps 29 may have guide rails that are movably mounted. This is beneficial as in some instances the femoral bone formation 11 may have an outer edge which deviates from straight and if one of the clamps is applied to such a deviation, this may cause the guide rails 17 to misalign. Therefore if one of the inner clamps 29 is applied to such a deviation, having a movable guide rail 17 facilitates the insertion of the guide rod 19.

In situations where the hip or other implant is not cemented in place, it may not be possible to remove the implant prior to application of the support 1 as described above. In such a case the support 1 are firstly applied to support the bone and are used as a guide while removing the implant.

It will be appreciated that the preferred embodiment and/or modified versions of the preferred embodiment can be applied to the insertion and extraction of a wide variety of implants and its uses are not limited to hip revision surgery. For example, a preferred embodiment of the present invention is adapted for use in the methods and with the apparatus disclosed in co-pending patent application no. PCT/AU02/01482 (WO 03/037192), the contents of which are hereby incorporated in their entirety by way of reference.

1. A bone support for use during removal of unwanted material from within a cavity of a bone formation during surgery, said support including a surgical support clamp adapted to engage an external surface of the bone formation, the clamp having a pair of elongated arms with opposed bone-supporting jaws at one end to engage the bone formation, a connecting arrangement by which the arms are connected together and are moveable relative to each other, and an arm moving arrangement associated with the arms by which the opposed jaws are adapted to be urged together to securely grip and support the bone formation.

2. A bone support for use in supporting a bone during a surgical operation in which unwanted material is removed from within a cavity of the bone formation, the support including one or more support clamps located by a guide, each support including engagement means to fit around and support said bone during said operation.

3. A bone support according to claim 1 wherein the or each support clamp includes a surgical tool guide means to guide a cutting tool for removing said unwanted material.

4. A bone support according to claim 3 wherein the or each support includes two arms connected at or adjacent their proximal ends for movement relative to each other, each of said arms having a respective one of a pair of opposed bone-supporting jaws at the distal end.

5. A bone support according to claim 1 wherein the arms are hinged together adjacent their proximal ends such that movement of the proximal ends away from each other causes resulting movement of the jaws towards each other.

6. A bone support according to claim 5 wherein said support clamp includes an adjustable arm moving arrangement for selectively moving said jaws into and out of gripping engagement with said bone.

7. A bone support according to claim 6 wherein said arm moving arrangement is located at or adjacent said proximal ends of said arms.

8. A bone support according to claim 7 wherein said arm moving arrangement includes a threaded shaft threadedly engaged through one said arm; a handle located at one end of said threaded shaft; and an abutment portion at the other end of said shaft such that relative rotation of said handle moves said abutment portion into engagement with the other said arm thereby moving said bone-support jaws into gripping engagement with said bone formation.

9. A bone support according to claim 8 wherein said jaws are substantially concave to fit closely around the outer surface of said bone formation.

10. A bone support according to claim 9 wherein a plurality of support clamps are positioned along said bone to support the bone along its length.

11. A bone support according to claim 10 wherein each support clamp is aligned relative to the longitudinal axis of said bone cavity.

12. A bone support according to claim 11 wherein said surgical tool guide means includes a guide rail mounted to at least one of said arms wherein said guide rail is adapted to receive a guide display for displaying the orientation of said bone.

13. A bone support according to claim 12 wherein said guide display is a guide rod slidably mounted to said guide rail.

14. A bone support according to claim 13 wherein said guide rail is movably mounted to at least one of said arms for facilitating insertion of said guide rod.

15. A bone support according to claim 13 wherein said guide rail is fixedly mounted to at least one of said arms.

16. A method of aligning a surgical tool with a bone cavity comprising the steps of engaging a plurality of bone supports as claimed in claim 1 with an external surface of the bone formation, engaging a guide rod with guide rails on each bone support such that the guide rod extends substantially parallel with the bone cavity whereby a surgical tool is able to be aligned with the bone cavity using the guide rod as an aligning guide.

17. A surgical chisel for use in the method of claim 16, said chisel including a shaft having a hollow portion adjacent a cutting end; an abutment portion at the other end of said shaft; said hollow portion having tapering internal walls extending inwardly towards a central axis of the shaft to define a cutting edge at said cutting end.

18. A surgical chisel according to claim 17 wherein said hollow portion extends along a major portion of the length of said shaft.

19. A surgical chisel according to claim 18 wherein said shaft is generally circular in cross section.

20. A method of performing hip revision surgery on a patient, said method including the steps of:
a) exposing a femoral bone formation of said patient;
b) applying at least one bone support as claimed in claim 1 to said femoral bone formation to support the structure of femoral formation; and
c) removing unwanted material from within the cavity of said femoral bone formation.

21. A method according to claim 20 further including the step of:
d) extracting a first implant from within said femoral bone formation.

22. A method according to claim 21 further including the step of:
e) inserting a second implant into said femoral bone formation after removal of the unwanted material.

23. A method according to claim 22 within step (a) includes exposing said femoral bone of said patient through at least two incisions adjacent said bone.

24. A method according to claim 23 wherein step (b) includes applying the jaws of at least two bone supports to said femoral bone formation through said at least two incisions adjacent said bone.

25. A method according to claim 23 further including the step of engaging a guide rod with guide rails associated with each bone support such that the guide rod is substantially aligned with the bone cavity to guide the use of a surgical chisel to remove unwanted material from the cavity.

26. A bone support according to claim 2 wherein the or each support clamp includes a surgical tool guide means to guide a cutting tool for removing said unwanted material.

27. A bone support according to claim 4 wherein the arms are hinged together adjacent their proximal ends such that movement of the proximal ends away from each other causes resulting movement of the jaws towards each other.

28. A method of aligning a surgical tool with a bone cavity comprising the steps of engaging a plurality of bone supports as claimed in claim 2 with an external surface of the bone formation, engaging a guide rod with guide rails on each bone support such that the guide rod extends substantially parallel with the bone cavity whereby a surgical tool is able to be aligned with the bone cavity using the guide rod as an aligning guide.
